(19) United States
(12) Patent Application Publication
(10) Pub. No.: US 2008/0183182 A1
(43) Pub. Date: Jul. 31, 2008

(54) Guide wire

(76) Inventors: Hideo Satou, Fujinomiya-shi (JP); Hideki Fujimagari, Fujinomiya-shi (JP); Fumihiko Mouri, Fujinomiya-shi (JP)

Correspondence Address: BUCHANAN, INGERSOLL & ROONEY PC, POST OFFICE BOX 1404, ALEXANDRIA, VA, 22313-1404, US

(21) Appl. No.: 12/005,283

(22) Filed: Dec. 27, 2007

Related U.S. Application Data
(60) Provisional application No. 60/878,658, filed on Jan. 5, 2007.

(30) Foreign Application Priority Data
Dec. 28, 2006 (JP) 2006-356644

(51) Int. Cl. A61M 25/09

(52) U.S. Cl. 606/108; 604/164.13; 600/585

(57) ABSTRACT

A guide wire comprises a wire body including a filamentous first wire disposed on the distal side and comprised of a Ni—Ti alloy, and a filamentous second wire disposed on the proximal side of the first wire and comprised of a material higher in rigidity than the material constituting the first wire, with the first and second wires being connected to each other. The guide wire preferably includes a flexible member which is flexible and which covers the outer periphery of a portion, on at least the distal side, of the wire body. In the guide wire, a boundary portion between the proximal portion of the first wire and the distal portion of the second wire is located inside the flexible member.

This application claims priority under 35 U.S.C. § 119(e) to U.S. Provisional Application No. 60/878,658 filed on Jan. 5, 2007, the entire content of which is incorporated herein by reference. This application is also based on and claims priority to Japanese Application No. 2006-356644 filed on Dec. 28, 2006, the entire content of which is incorporated herein.

### TECHNOLOGICAL FIELD

The present invention generally relates to a guide wire, and more particularly pertains to a guide wire used in introducing a catheter into a body lumen such as a blood vessel and a bile duct.

### BACKGROUND DISCUSSION

Guide wires are used to guide a catheter in treatment of sites at which open surgeries are difficult or which require minimal invasiveness to the body, for example, PTCA (Percutaneous Transluminal Coronary Angioplasty), or during examination such as cardioangiography. A guide wire used in the PTCA procedure is inserted, with its distal end projecting from the distal end of a balloon catheter, into the vicinity of a target angiostenosis portion together with the balloon catheter, and is operated to guide the distal portion of the balloon catheter into the vicinity of the target angiostenosis portion.

In PTA (Percutaneous Transluminal Angioplasty), also, for opening a stenosis portion (occluded portion) formed in a peripheral blood vessel such as femoral, iliac, renal and shunt blood vessels, a distal portion of a balloon catheter is guided to the vicinity of an angiostenosis portion by use of a guide wire, like in the PTCA procedure.

Since the blood vessels to which such treating method is performed are bent in a complicated manner, a guide wire used to insert a balloon catheter into the blood vessel is required to have appropriate flexibility and resilience against bending, pushability and torque transmission performance (generically called “steerability”) for transmitting an operational force from the proximal portion to the distal side, and further kink resistance (resistance against sharp bending) and the like.

Guide wires include those in which, for realizing a structure having not only appropriate steerability but also appropriate flexibility at the distal portion of the guide wire, the guide wire is formed from different materials. More specifically, the guide wire has a first wire including a Ni—Ti alloy and a second wire including stainless steel.

However, the guide wire in which the joint portion between the first wire and the second wire is located on the proximal side relative to a coil, has had the following problem. For example, in the case of treating CTO (Chronic Total Occlusion) generated in a more complicatedly bent blood vessel, the torque exerted on the second wire may be reduced at that portion of the first wire which is not covered with the coil, and the torque may not be sufficiently transmitted to the coil (the distal portion of the guide wire).

### SUMMARY

According to one aspect, a guide wire comprises a wire body including a first wire disposed on the distal side and comprised of a Ni—Ti alloy, and a second wire disposed on the proximal side of the first wire and comprised of a material higher in rigidity than the material constituting the first wire. The first and second wires are connected to each other. The guide wire may include a flexible member covering the outer periphery of a portion of the wire body on at least the distal side of the wire body. The flexible member possesses flexibility and is comprised of a tubular body having an inner diameter.

The boundary portion preferably is provided with a projected portion projecting in a radially outward direction of the wire body. The outer diameter of the projected portion preferably is smaller than the inner diameter of the flexible member. A portion of the first wire covered with the flexible member can be longer than the portion of the second wire covered with the flexible member. Alternatively, the portion of the first wire covered with the flexible member is shorter than the portion of the second wire covered with the flexible member. The flexible member preferably is a tubular body. The flexible member may be coil formed by spirally winding a filamentous member. The tubular body is preferably provided with a groove and/or a slit in a wall portion. The flexible member can be comprised of two component parts arranged along the longitudinal direction of the wire body. The two component parts preferably are each a coil formed by spirally winding a filamentous member. One of the two component parts may be a coil formed by spirally winding a filamentous member, and the other is a metal pipe body. The two component parts can be comprised of the same metallic material or different metallic materials. The boundary portion preferably is located on the side of one of the two component parts which is disposed on the proximal side. The wire body may have a configuration in which the proximal end face of the first wire and the distal end face of the second wire are joined to each other. The joining of the proximal end face of the first wire and the distal end face of the second wire to each other preferably is conducted by welding.

According to another aspect, a guide wire includes a wire body including a filamentous first wire disposed on the distal side and having an alloy including Ni and Ti, and a second wire disposed on the proximal side of the first wire and having a material higher in rigidity than the material constituting the first wire, the first and second wires connected to each other. The guide wire can include a flexible member possessing flexibility and covering the outer periphery of a portion of the wire body on at least the distal side. The guide wire can include a boundary portion between the proximal portion of the first wire and the distal portion of the second wire that is located on the inside of the flexible member. The wire body may have a pipe-like connecting member for connection between a proximal portion of the first wire and a distal portion of the second wire. At least a portion of the connecting member preferably is supported relative to the flexible member. A distal portion and/or a proximal portion of the connecting member preferably is supported relative to the flexible member. An intermediate portion of the connecting member preferably is supported relative to the flexible member.

According to another aspect, a guide wire comprises a wire body possessing an outer peripheral surface and comprised of a filamentous first wire and a filamentous second wire, with the second wire positioned proximally of the first wire. The first wire comprises a proximal end portion terminating proximally in a proximal end face, and the second wire comprises a distal end portion terminating distally in a distal end face. The proximal end portion of the first wire possesses an outer diameter that is constant to the proximal end face to define a constant outer diameter proximal end portion of the first wire, and the distal end portion of the second wire possesses an outer diameter that is constant to the distal end face to define a constant outer diameter distal end portion of the second wire. The first wire is comprised of an alloy including Ni and Ti, the second wire is comprised of a material different from the material constituting the first wire, with the material constituting the second wire being different in rigidity than the material constituting the first wire, and the proximal end face of the first wire abuts and is joined to the distal end face of the second wire. A flexible member covers the outer peripheral surface of a portion of the wire body so that the entirety of the constant outer diameter proximal end portion of the first wire is positioned inside the flexible member and at least a part of the constant outer diameter distal end portion of the second wire is positioned inside the flexible member. The outer peripheral surface of the constant outer diameter proximal end portion of the first wire is spaced from the inner surface of the flexible member, and the outer peripheral surface of the part of the constant outer diameter distal end portion of the second wire is spaced from the inner surface of the flexible member. A first fixing member fixes the distal end of the flexible member to the wire body, a second fixing member fixes the proximal end of the flexible member to the wire body, and a third fixing member fixes the intermediate portion of the flexible member to the wire body. The length of the constant outer diameter proximal end portion of the first wire that is positioned inside the flexible member is different from the length of the part of the constant outer diameter distal end portion of the second wire that is positioned inside the flexible member.

### BRIEF DESCRIPTION OF THE DRAWING FIGURES

The foregoing and additional features and aspects of the guide wire will become more apparent from the following detailed description considered with reference to the accompanying drawing figures briefly described below.

FIG. 1 is a longitudinal cross-sectional view of a first embodiment of the guide wire disclosed herein.

FIG. 2 is a longitudinal cross-sectional view of a second embodiment of the guide wire disclosed herein.

FIG. 3 is an enlarged longitudinal cross-sectional view of the distal portion of a wire body in a guide wire according to a third embodiment.

FIG. 4 is an enlarged longitudinal cross-sectional view of the distal portion of a wire body in a guide wire according to a fourth embodiment.

FIG. 5 is an enlarged longitudinal cross-sectional view of the distal portion of a wire body in a guide wire according to a fifth embodiment disclosed herein.

FIG. 6 is an enlarged longitudinal cross-sectional view of the distal portion of a wire body in a sixth embodiment of n a guide wire disclosed herein.

FIG. 7 is an enlarged longitudinal cross-sectional view of the distal portion of a wire body in a seventh disclosed embodiment of a guide wire.

FIG. 8 is an enlarged longitudinal cross-sectional view of the distal portion of a wire body in a guide wire according to an eighth embodiment.

FIG. 9 is a plan view of a flexible member in the guide wire shown in FIG. 8.

FIG. 10 is an enlarged longitudinal cross-sectional view of a distal portion of a wire body in a guide wire according to a ninth embodiment.

FIG. 11 is an enlarged longitudinal cross-sectional view of the distal portion of a wire body in a tenth embodiment of a guide wire disclosed herein.

### DETAILED DESCRIPTION

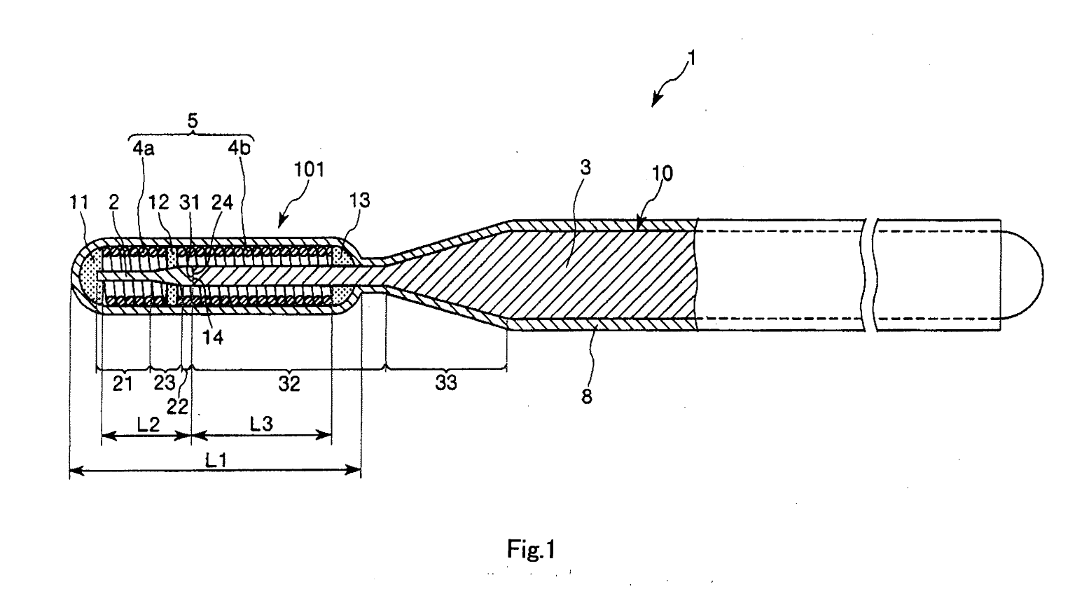

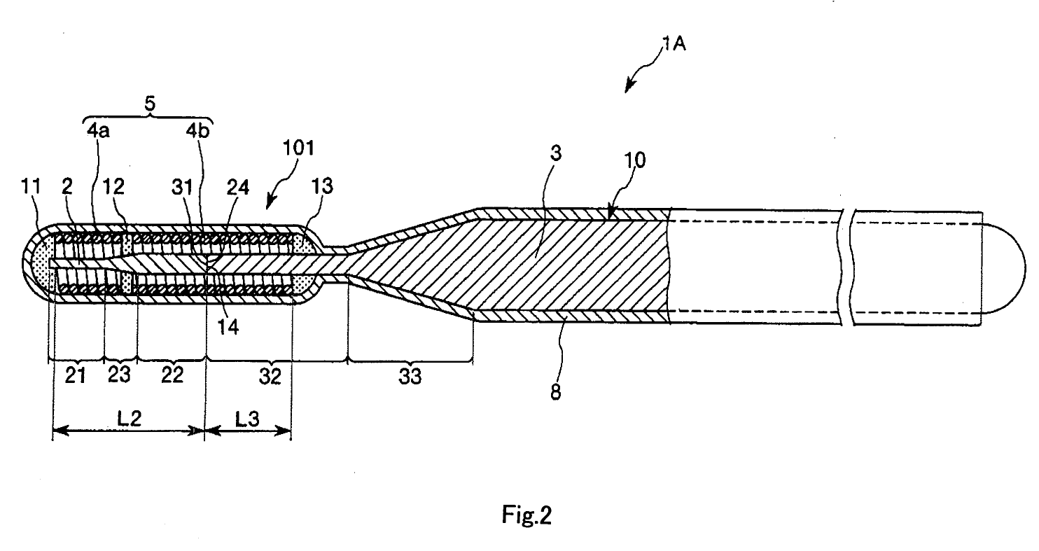

FIG. 1 illustrates a first embodiment of the guide wire disclosed herein. The right side in FIG. 1, as well as in FIGS. 2-11, is referred to as the “proximal” side while the left side is referred to as the “distal” side. In addition, in FIG. 1 (as well as FIGS. 2-11), to help facilitate an understanding, the guide wire is schematically shown in the state of being shortened in the longitudinal direction and exaggerated in the radial (diametrical) direction, so that the ratio between the dimensions in the longitudinal direction and in the radial direction is different from the practical or actual ratio.

The guide wire shown in FIG. 1 is a catheter guide wire adapted to be inserted in the lumen of a catheter (inclusive of endoscope). The guide wire 1 includes a wire body 10 in which a first wire 2 disposed on the distal side and a second wire 3 disposed on the proximal side of, and adjacent to, the first wire 2 are joined (connected) to each other by welding, and a flexible member 5 covering the outer periphery of a distal-side portion (distal portion 101) of the wire body 10.

The overall length of the guide wire 1 is not particularly limited, and is preferably about 200 to 5,000 mm. In addition, the length L of the distal portion 101, depending on the overall length of the guide wire 1, is preferably about 250 to 300 mm.

The first wire 2 includes a flexible or elastic filamentous member. Examples of the material constituting the filamentous member (first wire 2) include an alloy including Ni and Ti, for example Ni—Ti alloys such as a Ni—Ti alloy containing 49 to 52 at. % of Ni. The Ni—Ti alloys are comparatively flexible, have resilience and are less liable to acquire a tendency toward a certain bending. Therefore, with the first wire 2 including a Ni—Ti alloy, the guide wire 1 can have sufficient flexibility and resilience against bending at its distal-side portion, so that trackability in relation to complicatedly curved or bent blood vessels is enhanced, and improved steerability can be obtained. In addition, the resilience of the first wire 2 prevents the first wire 2 from acquiring a tendency toward a certain bending or set even when the first wire is repeatedly curved or bent, so that it is possible to prevent the steerability from being lowered due to a tendency toward a certain bending or set which might otherwise be acquired by the first wire 2 during use of the guide wire 1.

The Ni—Ti alloys having the above-mentioned composition can be made to have superelasticity by a heat treatment or the like. However, even Ni—Ti alloys which contain more than 52 at. % of Ni and do not substantially exhibit superelasticity can also be used insofar as they have appropriate flexibility and elasticity.

The first wire 2 comprises constant-outer-diameter portions 21, 22 which have a constant outer diameter, and a tapered gradually reduced outer diameter portion 23 located between the constant-outer-diameter portions 21, 22 and of which the outer diameter is gradually reduced along the distal direction.

The gradually reduced outer diameter portion 23 of the first wire 2 helps ensure that the rigidity (flexural rigidity, torsional rigidity) of the first wire 2 is gradually lowered or reduced along the distal direction. As a result, the guide wire 1 has good flexibility at its distal portion 101, whereby trackability in relation to blood vessels and the like, and safety, can be enhanced, and kinking (sharp bending) and the like can be prevented from occurring.

The taper angle (outer diameter reduction rate) of the gradually reduced outer diameter portion 23 may be constant along the longitudinal direction of wire body or may vary along the longitudinal direction at some portion. For example, a configuration may be adopted in which portions with a comparatively larger taper angle (outer diameter reduction rate) and portions with a comparatively smaller taper angle are alternately repeated a plurality of times.

In the constant-outer-diameter portion 21 located on the proximal side of the gradually reduced outer diameter portion 23, the outer diameter is constant over a range to the distal-most end of the first wire 2.

In the constant-outer-diameter portion 22 located on the proximal side of the gradually reduced outer diameter portion 23, the outer diameter is also constant over a range to the proximal-most end of the first wire 2, like the constant-outer-diameter portion 21.

While the number of the gradually reduced outer diameter portion(s) 23 in the illustrated embodiment shown in FIG. 1 is one, this configuration is not limitative, and, for example, the wire body can be configured to include two or more gradually reduced outer diameter portions. In addition, while the number of constant-outer-diameter portions is two in the illustrated embodiment shown in FIG. 1, this configuration is not limitative, and the number may be one or three or more, for example.

The distal end (distal end face 31) of the second wire 3 is connected (coupled) to the proximal end (proximal end face 24) of the first wire 2 by, for example, welding. The second wire 3 includes a filamentous member which is flexible or elastic like the first wire 2 and which is higher in rigidity than the material (Ni—Ti alloy) constituting the first wire 2. The material constituting the filamentous member (second wire 3) is not particularly limited, and examples of the material include various metallic materials such as stainless steel and cobalt alloys.

Examples of the stainless steel include all SUS steels such as SUS304, SUS303, SUS316, SUS316L, SUS316J1, SUS316J1L, SUS405, SUS430, SUS434, SUS444, SUS429, SUS430F, and SUS302.

The cobalt alloys may be any alloys that contain Co as a constituent element, but are preferably those containing Co as a main constituent (Co-based alloy, i.e., alloys in which the Co content is the highest of the contents of elements constituting the alloy), and are more preferably Co—Ni—Cr alloys. The cobalt alloys have high elasticity when formed into a wire, and have an appropriate elastic limit. Therefore, the second wire 3 including a cobalt alloy is excellent in torque transmission performance, and is extremely less liable to suffer buckling or the like problem. In addition, the cobalt alloys are high in elastic modulus, and can be cold worked even when they are made to have a high elastic limit. The high elastic limit helps ensure that the second wire 3 can be reduced in size while sufficiently preventing the generation of buckling, and it is possible to obtain a guide wire having sufficient flexibility and rigidity for insertion into a predetermined site.

Preferable examples of the Co—Ni—Cr alloys include alloys containing 28 to 50 wt. % of Co, 10 to 30 wt. % of Ni, 10 to 30 wt. % of Cr, and the balance of Fe, and alloys obtained by replacing part of these elements with other elements (substituent elements). When a substituent element or elements are contained in the alloy, an effect or effects intrinsic of the kind(s) of the substituent element(s) will be exhibited. For example, when at least one element selected from among Ti, Nb, Ta, Be, and Mo is contained in the alloy as a substituent element, the second wire 3 can be made to have a further enhanced strength or the like. Incidentally, where elements other than Co, Ni and Cr are contained in the alloy, the total content of all of them (all the substituent elements) is preferably not more than 30 wt. %.

Part of Co, Ni and Cr may be replaced with other element(s). For example, part of the Ni may be replaced with Mn. This helps promote a further improvement in workability, for example. Also, part of the Cr may be replaced with Mo and/or W. This permits a further improvement in elastic modulus, for example. Among the Co—Ni—Cr alloys, those which contain Mo, i.e., Co—Ni—Cr—Mo alloys are particularly preferable.

As mentioned above, the second wire 3 comprises a constant-outer-diameter portion 32 having a constant outer diameter, and a tapered portion 33 located on the proximal side of the constant-outer-diameter portion 32 and which possesses an outer diameter that is gradually increased along the proximal direction. On the proximal side of the tapered portion 33, the outer diameter of the second wire 3 is substantially constant along the longitudinal direction of wire.

The constant-outer-diameter portion 32 has a constant outer diameter over the range from the distal end of the constant-outer-diameter portion 32 to the proximal end of the constant-outer-diameter portion 32. The outer diameter of the constant-outer-diameter portion 32 is equal to the outer diameter of the proximal end face 24 (constant-outer-diameter portion 22) of the first wire 2.

The tapered portion 33 of the second wire 3 is located on the proximal side relative to the distal portion 101 (flexible member 5) having a length L1. The presence of the tapered portion 33 helps ensure a smooth variation in physical properties, particularly elasticity from the second wire 3 to the first wire 2.

As mentioned above, the first wire 2 has the constant-outer-diameter portion 22 on the most proximal side thereof, and the second wire 3 has the constant-outer-diameter portion 32 on the most distal side thereof. In other words, the outer diameter of the wire body 10 is substantially constant respectively on the front side and the rear side (in the distal-side vicinity and in the proximal-side vicinity) of the joint surface (boundary portion, weld portion) 14 between the first wire 2 and the second wire 3 as illustrated in FIG. 1.

As a result, flexibility and resilience against bending in the vicinity of the joint surface 14 can be sufficiently secured.

In the guide wire 1, the average outer diameter of the first wire 2 is smaller than the average outer diameter of the second wire 3. This helps ensure that the guide wire 1 is rich in flexibility at the first wire 2 on the distal side and comparatively high in rigidity at the second wire 3 on the proximal side, so that both flexibility at the distal portion and excellent steerability (pushability, torque transmission performance, etc.) can be realized simultaneously. The average outer diameter of the first wire and the second wire can be obtained or determined by the average of the outer diameter measurements at five spaced apart locations on the respective wires 2, 3, including at least one in each of the sections 21, 22, 23 for the first wire and at least one in each of the sections 32, 33 for the second wire.

The first wire 2 and the second wire 3 constituting the wire body 10 are connected and fixed to each other by welding as mentioned above. As a result, a high joint strength is obtained at the joint surface (weld portion) 14 between the first wire 2 and the second wire 3 by a relatively simple method. The particular method for welding the first wire 2 and the second wire 3 is not limited. Examples of the welding method which can be used include friction welding, spot welding by use of laser, butt resistance welding such as upset welding, and the like. Among the different methods, butt resistance welding is particularly preferred from the viewpoint of the comparatively easy process thereof and a high joint strength obtained thereby.

The flexible member 5 is disposed at the distal portion 101 of the wire body 10. The flexible member 5 possesses flexibility and covers the outer periphery of the region of the wire body at which is located the joint surface 14 so as to be astride the joint surface 14 (i.e., extend on either side of the joint surface 14). Thus, the flexible member 5 has a longitudinal extent covering the entire first wire 2 and the constant-outer-diameter portion 32 of the second wire 3. The flexible member 5 includes two coils (component parts) 4a, 4b disposed along the longitudinal direction of the wire body 10 and possessing different lengths. The coil 4a disposed on the distal side is shorter than the coil 4b disposed on the proximal side.

The coils 4a, 4b are each formed by spirally winding a filamentous member (thin wire). As a result, the flexible member 5 is a tubular body. The distal portion 101 of the wire body, in the vicinity of the joint surface 14, is positioned inside the tubular body (flexible member 5) so that the joint surface 14 is in a substantially central portion of the inside of the flexible member 5. In addition, the distal portion 101 is positioned in such a way that its outer surface is spaced from and does not make contact with the inside surface of the flexible member 5. That is, a space is formed between the outside surface of the distal portion 101 of the wire body and the inside surface of the flexible member 5.

In the configuration shown in FIG. 1, the coils 4a, 4b may be constructed so that with no external force exerted thereon, a gap exists between the adjacent turns of the spirally wound filamentous member. Alternatively, the coils 4a, 4b may be constructed so that with no external force exerted thereon, the coils are closely wound so as not to have any gap between the adjacent turns of the spirally wound filamentous member. As a further alternative, one of the coils can be constructed so that with no external force exerted thereon a gap exists between the adjacent turns of the spirally wound filamentous member while the other coil is constructed so that with no external force exerted thereon the coil is closely wound without any gaps between the adjacent turns of the spirally wound filamentous member.

The coils 4a, 4b each preferably include metallic materials. Examples of the material include not only stainless steels, superelastic alloys, and cobalt alloys but also noble metals such as gold, platinum, tungsten, etc. and alloys containing such noble metals (for example, platinum-iridium alloy). Especially where the coils include a radiopaque material such as a noble metal, the guide wire 1 can have a fluoroscopic imageability, so that the guide wire 1 can be inserted into the body while fluoroscopically confirming the position of the distal portion 101.

The coils 4a, 4b may be fabricated of the same metallic material or may be made of different metallic materials. Where the same constituent material is used, the number of kinds of constituent materials to be used is reduced, so that the manufacturing cost of the guide wire 1 is not excessively high. Where different constituent materials are used, it is possible, for example, to appropriately modify the overall physical properties of the flexible member 5. In the latter case, it is preferable, for example, that the coil 4a includes a radiopaque material (a noble metal or the like) and the coil 4b includes a comparatively radiolucent material (stainless steel or the like).

The flexible member 5 is fixed to the wire body 10 at a distal portion of the coil 4a, at a position between the coil 4a and the coil 4b, and at a proximal portion of the coil 4b, by fixing materials 11, 12 and 13, respectively.

More specifically, the coil 4a is fixed to a distal portion of the constant-outer-diameter portion 21 of the first wire 2 by the fixing material 11, and is fixed at the gradually reduced outer diameter portion 23 of the first wire 2 by the fixing material 12. In addition, the coil 4b is fixed at the gradually reduced outer diameter portion 23 of the first wire 2 (together with the coil 4a) by the fixing material 12, and is fixed to a proximal portion of the constant-outer-diameter portion 32 of the second wire 3 by the fixing material 13.

These fixing materials 11, 12, 13 each includes solder (brazing filler). The respective fixing materials 11, 12, 13 are not limited to solder, and may instead be an adhesive. The method of fixing the flexible member 5 (coils 4a, 4b) is also not limited to the use of the fixing material. For example, welding may also be used to effect the fixing. In addition, to prevent damage to the inside wall of a body lumen such as a blood vessel, the distal end surface of the fixing material 11 is preferably rounded in shape.

With such a flexible member 5 disposed, the guide wire 1 can be reduced in the area of contact with the lumen because the distal portion 101 is covered with the flexible member 5, so that sliding resistance can be reduced. Accordingly, the steerability of the guide wire 1 is further enhanced.

As shown in FIG. 1, the majority part (substantially the entire part) of the first wire 2 is positioned inside the flexible member 5, and the majority part (substantially the entire part) of the constant-outer-diameter portion 32 of the second wire 3 is positioned inside the flexible member 5. Therefore, the joint surface 14 between the first wire 2 and the second wire 3 is located on the inside of the flexible member 5.

As has been described above, the second wire 3 of the guide wire 1 includes a stainless steel or a cobalt alloy. Therefore, when a torque is exerted on the proximal portion (hand-operated portion) of the second wire 3, the torque is securely transmitted to the constant-outer-diameter portion 32. The torque thus transmitted to the constant-outer-diameter portion 32 is securely transmitted through the comparatively short first wire 2 to the distal end of the first wire 2. Thus, the guide wire 1 is excellent in torque transmission performance (steerability). In the case of treatment of CTO (Chronic Total Occlusion), for example, an operation (inserting operation) for inserting the distal portion 101 of the guide wire 1 to a diseased portion (stenosis portion) can be securely carried out owing to the excellent torque transmission performance of the guide wire 1.

The joint surface 14 is located on the inside of the flexible member 5, i.e., the flexible member 5 is so disposed as to be astride the joint surface 14. Therefore, variations in rigidity (flexural rigidity) generated at the joint surface 14 can be moderated by the flexible member 5. As a result, when the distal portion 101 of the guide wire 1 is inserted into a curved blood vessel, the guide wire 1 can be curved smoothly in the vicinity of the joint surface 14. Thus, steerability of the guide wire 1 is enhanced.

The first wire 2 including a Ni—Ti alloy is covered (protected) by the flexible member 5. Therefore, when the guide wire 1 is pushed in from the proximal side, the first wire 2 is inhibited from buckling under the pushing-in force exerted from the second wire 3 side. Thus, the guide wire 1 is excellent also in pushability.

As shown in FIG. 1, in the guide wire 1, the portion of the first wire 2 covered with the flexible member 5 (the portion with a length L2) is preferably shorter than the portion of the second wire 3 covered with the flexible member 5 (the portion with a length L3). This helps ensure that the overall rigidity of the distal portion 101 of the guide wire 1 is comparatively high. In addition, the proportion of the high-rigidity portion is comparatively high and so the torque transmission performance is enhanced.

The joint surface 14 is located on the inside of the flexible member 5 as mentioned above. It is particularly preferable that the joint surface 14 is located on the coil 4b side (i.e., that the joint surface 14 is surrounded by then coil 4b). This helps ensure that the area of fixation to the coil 4b is increased, and even if rupture at the joint surface 14 should occur, the first wire 2 and the second wire 3 can be favorably prevented from coming off.

In the illustrated embodiment, the flexible member 5 is comprised of two component parts, namely the two coils 4a, 4b. However, the guide wire is not limited in this regard. For example, the flexible member 5 may be comprised of a single coil having a length of L2+L3, or may include three or more coils having a total length of L2+L3.

In the described embodiment, the coils 4a, 4b are each formed by use of a filamentous member having a circular cross-sectional shape. However, the coils are not limited in this regard. The cross-sectional shape of the filamentous member used to form the coils 4a, 4b may be, for example, an ellipse, a tetragon (particularly, rectangle) or the like. Also, filamentous members having different cross-sectional shapes can be used to manufacture the two coils 4a, 4b.

As shown in FIG. 1, the guide wire 1 is provided on its outside surface with a resin coating layer 8 covering at least a part of the outside surface. In the illustrated embodiment, the resin coating layer 8 covers the entire outside surface. The resin coating layer 8 may be formed for any of various purposes. An exemplary purpose is to reduce the friction (sliding resistance) of the guide wire 1, thereby obtaining an enhanced slidability and enhancing the steerability of the guide wire 1.

In order to achieve a reduction in the friction (sliding resistance) of the guide wire 1, the resin coating layer 8 preferably includes a material which can reduce friction as will be described below. As a result, the frictional resistance (sliding resistance) between the guide wire 1 and the inside wall of a catheter used together with the guide wire 1 is reduced, slidability of the guide wire 1 is enhanced, and the steerability of the guide wire 1 in the catheter is improved. In addition, since the sliding resistance of the guide wire 1 is lowered, it is possible, when the guide wire is moved and/or rotated in a catheter, to reliably prevent kinking (sharp bending) or torsion of the guide wire 1, particularly, kinking or torsion in the vicinity of the joint surface 14.

Examples of the material which can be used to reduce friction include polyolefins such as polyethylene, polypropylene, polyvinyl chloride, polyesters (PET, PBT, etc.), polyamides, polyimides, polyurethane, polystyrene, polycarbonates, silicone resins, fluororesins (PTFE, ETFE, etc.), and composite materials thereof.

In addition, the resin coating layer 8 may be provided also for the purpose of enhancing the safety in inserting the guide wire 1 into a blood vessel or the like. For this purpose, it is preferable for the resin coating layer 8 to include a material rich in flexibility (a soft material or an elastic material).

Examples of the material rich in flexibility include polyolefins such as polyethylene, polypropylene, polyvinyl chloride, polyesters (PET, PBT, etc.), polyamides, polyimides, polyurethane, polystyrene, silicone resins, thermoplastic elastomers such as polyurethane elastomer, polyester elastomers, polyamide elastomers, various rubber materials such as latex rubbers, silicone rubbers, and composite materials obtained by combining two or more of these.

Incidentally, the resin coating layer 8 may be a single layer or a laminate of two or more layers.

The outside surface of at least the distal portion 101 of the guide wire 1 is preferably coated with a hydrophilic material. The hydrophilic material develops lubricity when wetted, whereby the friction (sliding resistance) of the guide wire 1 is reduced, and slidability thereof is enhanced. As a result, the steerability of the guide wire 1 is enhanced.

Examples of the hydrophilic material include cellulose based polymer materials, polyethylene oxide based polymer materials, maleic anhydride based polymer materials (for example, maleic acid copolymers such as methyl vinyl ether-maleic anhydride polymer), acrylamide based polymer materials (for example, polyacrylamide, polyglycidyl methacrylate-dimethylacrylamide (PGMA-DMAA) block copolymer), water-soluble nylon, polyvinyl alcohol, and polyvinyl pyrrolidone.

These hydrophilic materials, in many cases, exhibit lubricity by being wetted (absorbing water) so as to reduce the frictional resistance (sliding resistance) between the guide wire 1 and the inside wall of a catheter used together with the guide wire 1. This enhances the slidability of the guide wire 1, leading to enhanced steerability of the guide wire 1 in a catheter.

FIG. 2 is a longitudinal cross-sectional view of a second embodiment of the guide wire disclosed herein.

The following description of the second embodiment will center primarily upon the differences between this embodiment and the above-described embodiment. Features associated with the second embodiment that correspond to those in the first embodiment are designated with the same reference numeral, and a detailed description of such features is not repeated here.

This second embodiment is the same as the first embodiment above, except for the relationship in length between the portion of the first wire covered with the flexible member, and the portion of the second wire covered with the flexible member.

In the guide wire 1A shown in FIG. 2, the length L2 of the portion of the first wire 2 covered with the flexible member 5 is larger than the length L3 of the portion of the second wire 3 covered with the flexible member 5.

With this construction, the overall rigidity of the distal portion 101 of the guide wire 1A is reduced as compared with the overall rigidity of the distal portion 101 in the first embodiment above.

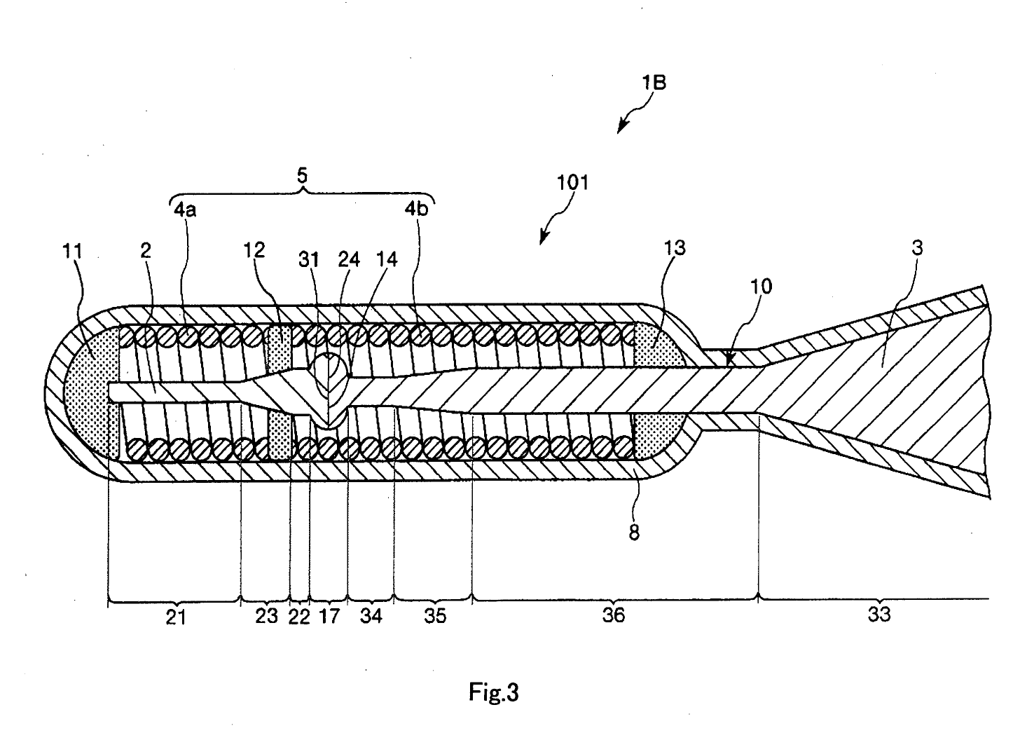

FIG. 3 is an enlarged longitudinal cross-sectional view of the distal portion of a wire body in a guide wire according to a third embodiment.

The following description of the third embodiment will focus primarily upon the differences between this embodiment and the above-described embodiments. Features associated with the third embodiment that correspond to those in the previously described embodiments are designated with the same reference numerals used in the earlier embodiments, and a detailed description of such features is not repeated here.

This third embodiment is the same as the first embodiment above, except for the shape of the guide wire in the vicinity of the joint surface.

In the guide wire 1B shown in FIG. 3, a projected portion 17 projecting in the outer peripheral direction (the radially outward direction) of the wire body 10 is formed at the joint surface 14. This projected portion 17 increases the area of the joint between the first wire 2 and the second wire 3, and the joint strength there is especially high. This helps provide that the torsional torque and a pushing-in force exerted from the second wire 3 in the guide wire 1 is more reliably transmitted to the first wire 2. As shown in FIG. 3, the outer diameter of the projected portion 17 is smaller than the inner diameter of the flexible member 5 so that the outer surface of the projected portion is spaced from the inner surface of the flexible member 5.

Such a projected portion 17 can be formed, for example, by a method in which at the time of welding the first wire 2 and the second wire 3 to each other by use of a butt welding machine, the wires are pressure contacted with each other so as to form a burr protruding in the radial direction (as the projected portion 17). That is, the wires are pushed axially towards one another with a force during the welding to result in the formation of a burr.

The second wire 3 is provided, on the proximal side relative to the projected portion 17, with a first constant-outer-diameter portion 34, a small tapered portion (tapered portion) 35, and a second constant-outer-diameter portion 36 in this order. As shown in FIG. 3, the entire first constant-outer-diameter portion 34 and the entire small tapered portion 35, and the majority of the second constant-outer-diameter portion 36, are located inside the flexible member 5.

The first constant-outer-diameter portion 34 is a portion whose outer diameter is constant along the longitudinal direction of wire and is smaller than the outer diameter of the constant-outer-diameter portion 22 of the first wire 2. In addition, the flexural rigidity of the first constant-outer-diameter portion 34 is substantially equal to the flexural rigidity of the constant-outer-diameter portion 22 on the proximal side of the first wire 2. The outer diameter of the first constant-outer-diameter portion 22 of the first wire 2 is greater than the outer diameter of the first constant-outer-diameter portion 34.

The small tapered portion 35 is a portion in which the outer diameter is gradually increased along the proximal direction. In addition, the small tapered portion 35 is set to be shorter in length than the tapered portion 33.

The second constant-outer-diameter portion 36 is a portion in which the outer diameter is constant along the longitudinal direction of the wire. The outer diameter of the second constant-outer-diameter portion 36 is equal to the outer diameter of the constant-outer-diameter portion 22 of the first wire 2.

The configuration of the guide wire 1B, including the first constant-outer-diameter 34 and the small tapered portion 35, provides a guide wire in which physical properties, particularly the elasticity, is varied smoothly from the second wire 3 to the first wire 2 so that excellent pushability and torque transmission performance are exhibited on the front (distal) and rear (proximal) sides of the joint surface 14 between the first wire 2 and the second wire 3, and kink resistance is also enhanced.

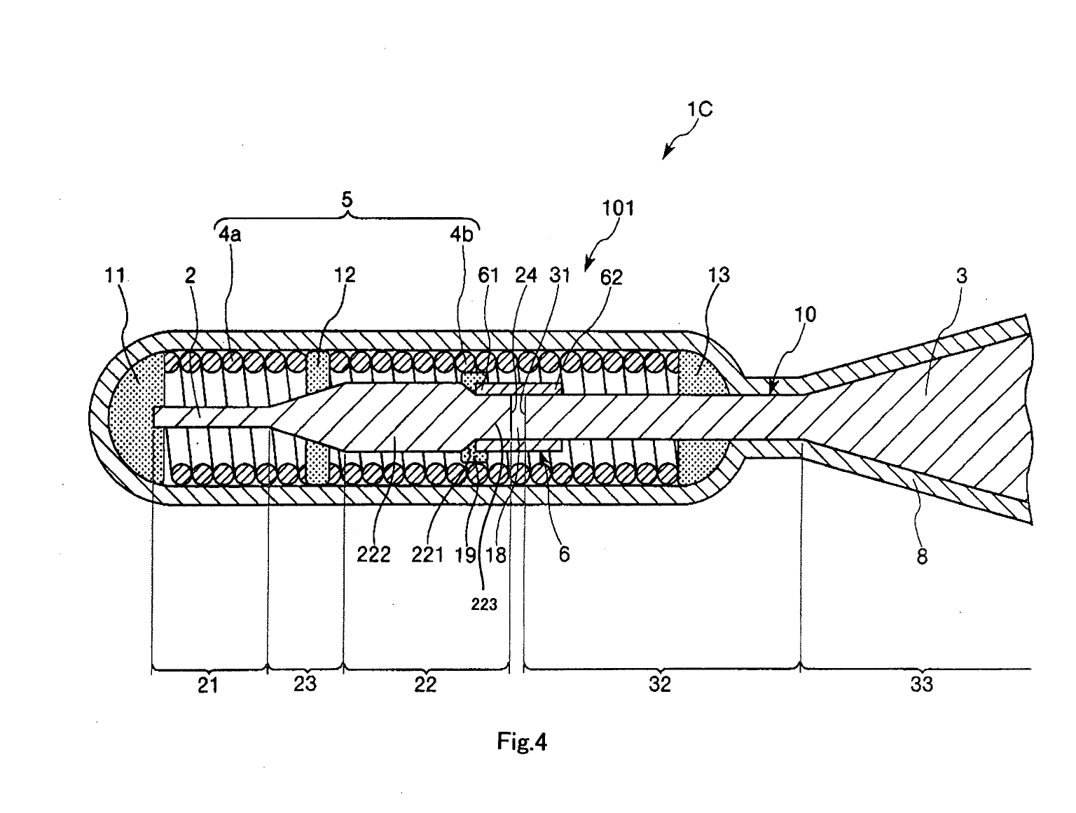

FIG. 4 is a longitudinal cross-sectional view, in an enlarged state, of the distal portion of a wire body in a guide wire according to a fourth embodiment.

The following description of the fourth embodiment primarily discusses differences between this embodiment and the above-described embodiments. Features associated with the fourth embodiment that correspond to those in the previously described embodiments are designated with the same reference numerals used in the earlier embodiments, and a detailed description of such features is not repeated here.

This fourth embodiment is the same as the first embodiment described above, except that the wire body in this embodiment further has a connecting member.

The wire body 10 in the guide wire 1C shown in FIG. 4 includes a connecting member 6 connecting the proximal portion of the first wire 2 and the distal portion of the second wire 3 to each other.

The connecting member 6 is pipe-like or cylindrical in shape. A constant-outer-diameter portion 22 of the first wire 2 is fitted into a distal portion 61 of the connecting member 6, and a constant-outer-diameter portion 32 of the second wire 3 is fitted into a proximal portion 62 of the connecting member 6, whereby the first wire 2 and the second wire 3 are reliably connected.

In the embodiment shown in FIG. 4, the distal portion 61 of the connecting member 6 is supportedly fixed to a flexible member 5 (coil 4b) through a fixing material 19. With the distal portion 61 thus fixed, a distal portion 101 of the wire body 10 can be fixed more firmly on the side of the first wire 2, which is high in flexibility, than on the side of the second wire 3, which is comparatively low in flexibility. In addition, the variation in rigidity is more gradual, which is advantageous in that the guide wire 1C can smoothly track the curvature of sharp bends or the like of a blood vessel.

The fixing material 19 includes, for example, a solder (brazing filler) or an adhesive.

The connecting member 6 preferably includes a metallic material. It is particularly preferable that the connecting member 6 includes the same Ni—Ti alloy as that constituting the first wire 2. Examples of the metallic material preferable for constituting the connecting member 6, other than the Ni—Ti alloy, include the same materials as those for the second wire 3, and Ni-based alloys.

The first wire 2 and the second wire 3 are not necessarily limited to constructions which permit fitting the respective end portions into the connecting member 6. For example, the first and second wires 2, 3 may be adhered to the connecting member 6 with an adhesive.

That portion of the first wire 2 which is connected to the connecting member 6 is not limited to having an outer diameter that is constant along its entire extent in the longitudinal direction of the wire. For example, the portion may be the constant-outer-diameter portion 22 provided with a stepped portion 221, as shown in FIG. 4. The stepped portion 221 is a portion where the outer diameter of a part of the constant-outer-diameter portion 22 is changed, i.e., gradually reduced along the proximal direction. Through the stepped portion 221, the constant-outer-diameter portion 22 can be divided into a distal-side portion 222 having a comparatively larger outer diameter and a proximal-side portion 223 shown in FIG. 4 that is smaller in outer diameter than the distal-side portion 222.

It is preferable that the rigidity of the portion 222 is substantially equal to the rigidity of the constant-outer-diameter portion 32 of the second wire 3. Such a relationship in rigidity is achieved, for example, by virtue of the outer diameter of the portion 222 being larger than the outer diameter of the constant-outer-diameter portion 32 as shown in FIG. 4.

In the guide wire 1C, the portion 223 is inserted into and connected to the connecting member 6. In addition, the fixing material 19 is present over a range covering both the distal portion 61 of the connecting member 6 and the stepped portion 221 of the constant-outer-diameter portion 22. The fixing material 19 fixes the first wire 2 and the coil 4b so as to fill up the gap between the stepped portion 221 and a distal portion of the connecting member 6. With this configuration, the boundary portion between the distal portion 61 of the connecting member 6 and the stepped portion 221 of the constant-outer-diameter portion 22 is reinforced, and the strength of the boundary portion can be enhanced.

The guide wire 1C may be so configured that, as shown in FIG. 4, the proximal end face 24 of the first wire 2 and the distal end face 31 of the second wire 3 are spaced from each other to form a gap 18 therebetween. In addition, the gap 18 may be filled up with an adhesive, for example. This makes it possible to obtain a relatively high joint strength between the first wire 2 and the second wire 3.

The guide wire shown in FIG. 4 is not limited to a configuration in which the proximal end face 24 of the first wire 2 and the distal end face 31 of the second wire 3 are spaced from each other as the end faces 24, 31 may abut one another.

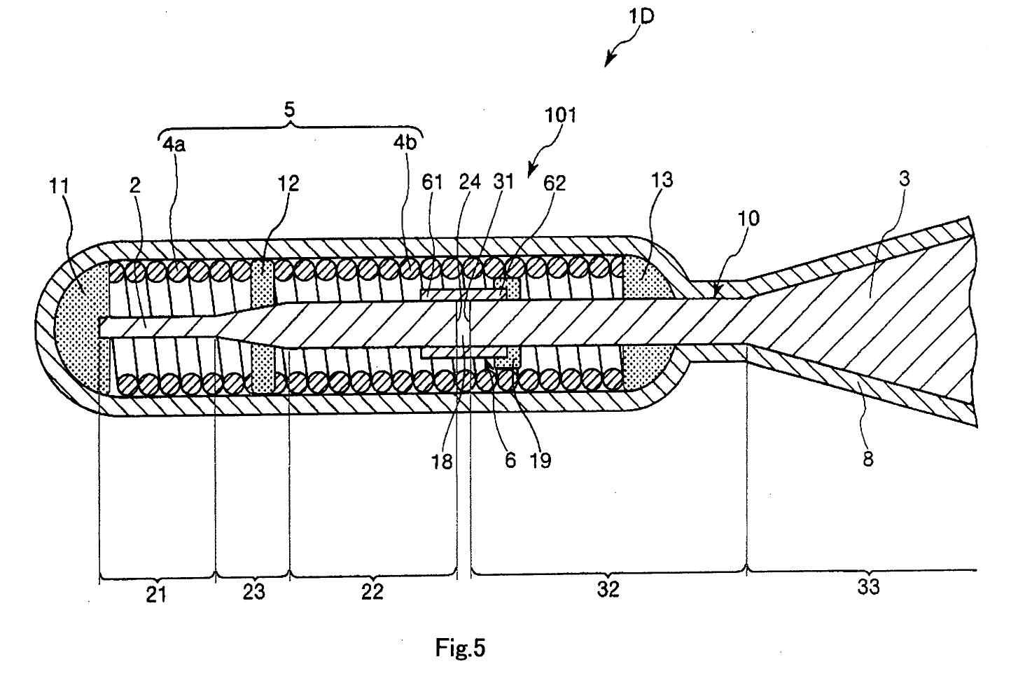

FIG. 5 is an enlarged longitudinal cross-sectional view of the distal portion of a wire body of a fifth embodiment of the guide wire.

The following description of the fifth embodiment primarily discusses differences between this embodiment and the above-described embodiments. Features associated with the fifth embodiment that correspond to those in the previously described embodiments are designated with the same reference numerals used in the earlier embodiments, and a detailed description of such features is not repeated here.

This fifth embodiment is the same as the fourth embodiment described above, except for the fixing position of the connecting member relative to the wire body.

With the connecting member 6 in the guide wire 1D shown in FIG. 5, the proximal portion 62 of the connecting member 6 is supportedly fixed to the flexible member 5 (coil 4b) through a fixing material 19. With the proximal portion 62 thus fixed, a distal portion 101 of the wire body 10 can be fixed more firmly on the side of the second wire, which is high in rigidity, than on the side of the first wire, which is comparatively low in rigidity. In addition, the variation in rigidity is more gradual, which is advantageous in that the guide wire 1D can smoothly track the curvature of sharp bends or the like of a blood vessel. Furthermore, in the case where the connecting member 6 includes a Ni—Ti alloy and the second wire 3 includes a stainless steel, the configuration in which the fixing material 19 covers the area ranging from the proximal portion 62 of the connecting member 6 to the surface of the second wire 3 makes it possible to supplement or increase the joint strength.

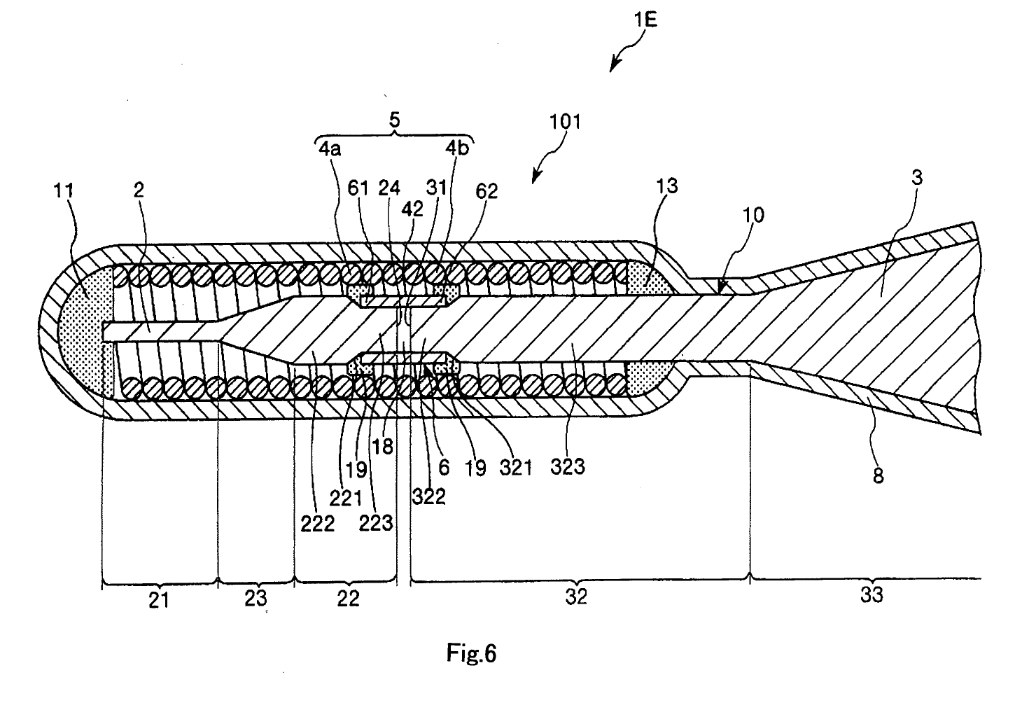

FIG. 6 is an enlarged longitudinal cross-sectional view of the distal portion of a wire body in a guide wire according to a sixth embodiment.

The sixth embodiment of the guide wire according to the present invention will be described below referring to this figure. The following description will be centered on the differences of this embodiment from the embodiments described above, and descriptions of the same items as above will be omitted.

This sixth embodiment is the same as the fourth embodiment above, except for the fixing position of the connecting member relative to the wire body.

The connecting member 6 in the guide wire 1E shown in FIG. 6 includes a distal portion 61 and a proximal portion 62 that are supportedly fixed to the flexible member 5 (coil 4b) respectively through fixing materials 19. With both the distal portion 61 and the proximal portion 62 thus fixed to the flexible member 5, the connecting member 6 is fixed more firmly (assuredly) than the connecting members 6 in the first and second embodiments. In addition, the variation in rigidity is more gradual, which is advantageous in that the guide wire 1E can smoothly track the curvature of sharp bends or the like of a blood vessel.

That portion of the second wire 3 which is connected to the connecting member 6 is not limited to a portion having a constant outer diameter along the longitudinal direction of the wire. For example, the portion may be the constant-outer-diameter portion 32 provided with a stepped portion 321 as shown in FIG. 6. The stepped portion 321 is a portion where the outer diameter of a part of the constant-outer-diameter portion 32 is changed, i.e., gradually increased along the proximal direction. Through the stepped portion 321, the constant-outer-diameter portion 32 can be divided into a distal-side portion 322 with a comparatively smaller outer diameter, and a proximal-side portion 323 smaller in outer diameter than the distal-side portion 222.

In the guide wire 1E, the portion 322 is inserted in and connected to the connecting member 6. In addition, of the two fixing materials 19, the fixing material 19 on the distal side is formed over a range covering the distal portion 61 of the connecting member 6 and the stepped portion 221 of the constant-outer-diameter portion 22. With this configuration, the boundary portion between a distal portion 61 of the connecting member 6 and a stepped portion 221 of the constant-outer-diameter portion 22 is reinforced, and the strength of the boundary portion is enhanced. The fixing material 19 on the proximal side is formed over a range covering a proximal portion 62 of the connecting member 6 and the stepped portion 321 of the constant-outer-diameter portion 32. With this configuration, the boundary portion between the proximal portion 62 of the connecting member 6 and the stepped portion 321 of the constant-outer-diameter portion 32 is reinforced, and the strength of the boundary portion is enhanced.

It is preferable that the rigidity of the portion 222 of the first wire 2 is substantially equal to the rigidity of the portion 322 of the second wire 3. This can be achieved, for example, by virtue of the outer diameter of the portion 222 of the first wire 2 being larger than the outer diameter of the portion 322 of the second wire 3.

In this embodiment, the rigidity of the portion 222 of the first wire 2 is lower than the rigidity of the portion 323 of the second wire 3. However, by constructing the outer diameter of the portion 222 to be greater than the outer diameter of the portion 323, it is possible to equalize the rigidity of the portion 222 and the rigidity of the portion 323.

In the guide wire 1E, the end portions of the coil 4a and the coil 4b are joined to each other by welding. The weld portion 42 is located between the fixing material 19 on the distal side and the fixing material 19 on the proximal side.

For example, in the case where the coils 4a, 4b include filamentous members of different materials (for example, a Pt—Ni alloy for one of the coils, and a stainless steel for the other) and they are welded to each other, the arrangement of the fixing materials 19 on both sides of the weld portion 42 makes it possible to prevent the weld portion 42 from being significantly influenced by the heat at the time of fixing the wire body 10 or the connecting member 6.

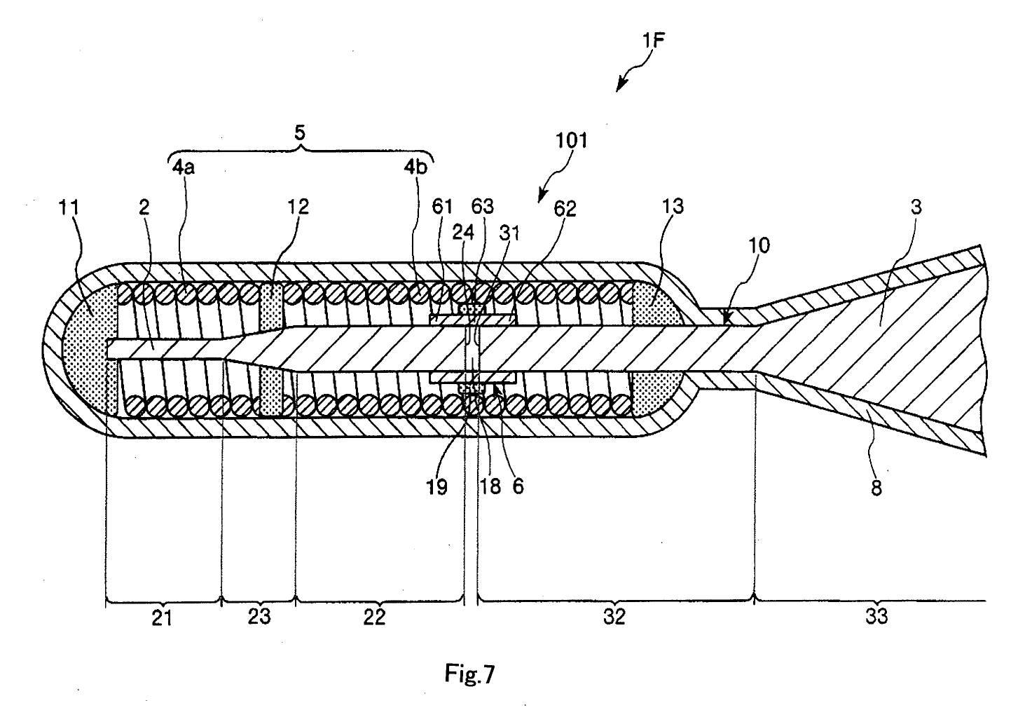

FIG. 7 is an enlarged longitudinal cross-sectional view of the distal portion of a wire body in a guide wire according to a seventh embodiment.

The following description of the seventh embodiment primarily discusses differences between this embodiment and the above-described embodiments. Features associated with the seventh embodiment that correspond to those in the previously described embodiments are designated with the same reference numerals used in the earlier embodiments, and a detailed description of such features is not repeated here.

This embodiment is similar to the fourth embodiment of the guide wire discussed above, except for the fixing position of the connecting member relative to the wire body.

The connecting member 6 in the guide wire 1F shown in FIG. 7 includes an intermediate portion, specifically a central portion 63, that is supportedly fixed to the flexible member 5 (coil 4b) through a fixing material 19. With the central portion 63 thus fixed, when the distal portion 101 of the guide wire 1F is inserted into a curved blood vessel, the connecting member 6 (the vicinity of the boundary portion between the first wire 2 and the second wire 3) can be curved relatively easily and stably. In addition, tensile strength is enhanced as is safety.

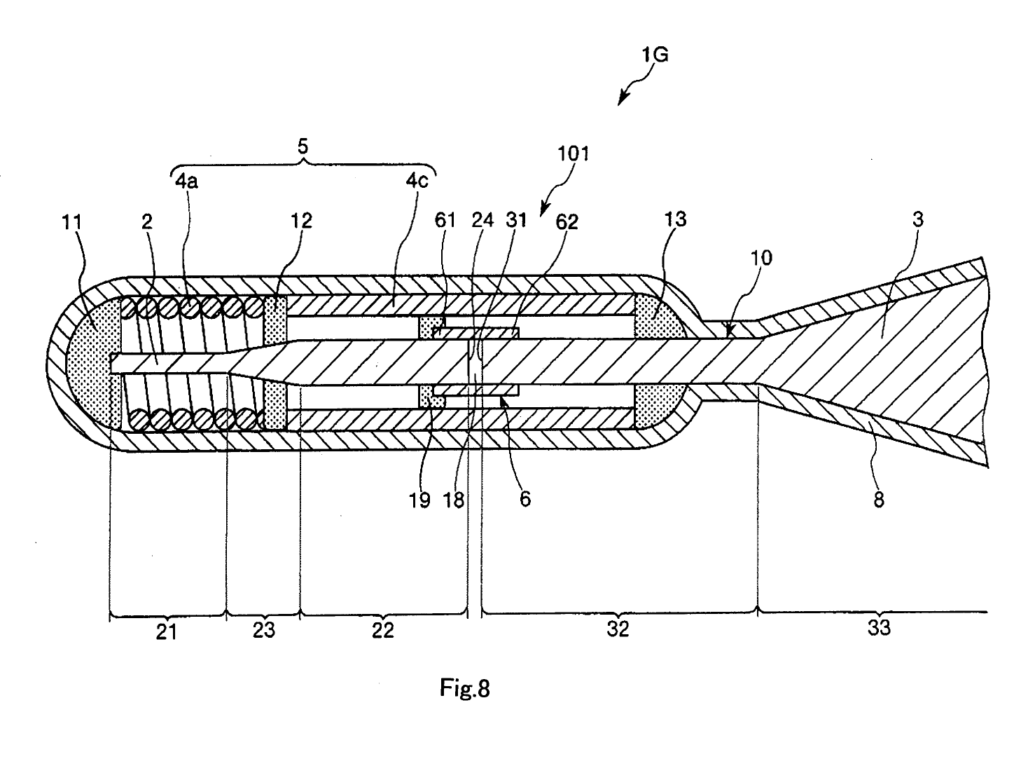

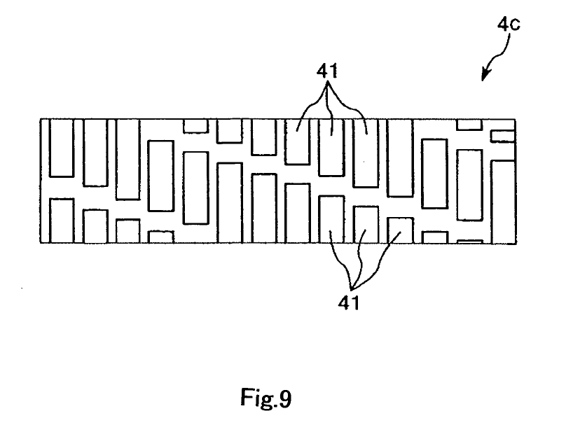

FIG. 8 is an enlarged longitudinal cross-sectional view of the distal portion of a wire body in an eight embodiment of a guide wire disclosed herein, while FIG. 9 is a plan view of the flexible member used in the guide wire shown in FIG. 8.

The following description of the eighth embodiment primarily discusses differences between this embodiment and the above-described embodiments. Features associated with the eighth embodiment that correspond to those in the previously described embodiments are designated with the same reference numerals used in the earlier embodiments, and a detailed description of such features is not repeated here.

This eighth embodiment of the guide wire is the same as the first embodiment described above and shown in FIG. 1, except for the configuration of the flexible member.

The flexible member 5 of the guide wire 1G shown in FIG. 8 includes two components, with the one on the proximal side comprising a tubular body 4c. The tubular body 4c is preferably a metal tubular body and constitutes a component part of the flexible member 5. The material constituting the tubular body 4c is not particularly limited. For example, the Ni—Ti alloys mentioned above in the description of the first wire 2 in the first embodiment above may be used, and Ni alloys and synthetic resin materials may also be used.

As shown in FIG. 9, the tubular body 4c is provided with a plurality of slits or grooves in a wall portion of the tubular body. In the illustrated embodiment, the tubular body includes slits 41. The slits (or grooves) 41 are each straight-line in shape, and extend in the circumferential direction of the tubular body 4c. In addition, the plurality of slits (or grooves) 41 are arranged along the circumferential direction and the longitudinal direction of the tubular body 4c. That is, the slits (or grooves) 41c possess a length (measured in the circumferential direction of the tubular body 4c) less than the circumference of the tubular body 4c so that a plurality of slits are spaced apart in the circumferential direction. In addition, slits positioned adjacent one another with respect to the longitudinal direction of the tubular body 4c are preferably staggered as illustrated in FIG. 9. Further, the slits are preferably of the same length in the circumferential direction of the tubular body 4c.

The plurality of slits 41 formed in the tubular body 4c impart greater flexibility to the tubular body 4c so that its flexural rigidity is reduced.

The illustrated configuration in which the plurality of slits 41 are the same in length along the circumferential direction of the tubular body 4c and are staggered in position along the circumferential direction, as shown in FIG. 9, is preferable because it enhances the isotropy of bendability.

With the tubular body 4c constructed in the manner described above, a torque exerted on the second wire 3 is effectively transmitted to the distal side through the tubular body 4c.

While the flexible member 5 is constructed in the illustrated embodiment with the coil 4a disposed on the distal side and the tubular body 4c disposed on the proximal side, the flexible member 5 is not limited in this regard. For example, a construction may be adopted in which the tubular body 4c is disposed on the distal side and the coil 4a is disposed on the proximal side.

While the flexible member 5 has a configuration in which only the component part on the proximal side includes the tubular body 4c, the flexible member 5 is not limited in this regard. For example, the flexible member 5 may be constructed so that the component part on the distal side also includes a tubular body 4c similarly to the tubular body on the proximal side.

While the illustrated version of the flexible member 5 includes one coil 4a and one tubular body 4c, the flexible member is also not limited in this regard. For example, the flexible member 5 may include one tubular body in which the length is equivalent to the total length of the coil 4a and the tubular body 4c.

The connecting member 6 is fixed on its distal side to the tubular body 4c of the first wire 2 through a fixing material 19 to impart excellent torque transmission performance to the guide wire. To help enhance the flexibility, the fixing material 19 may be omitted.

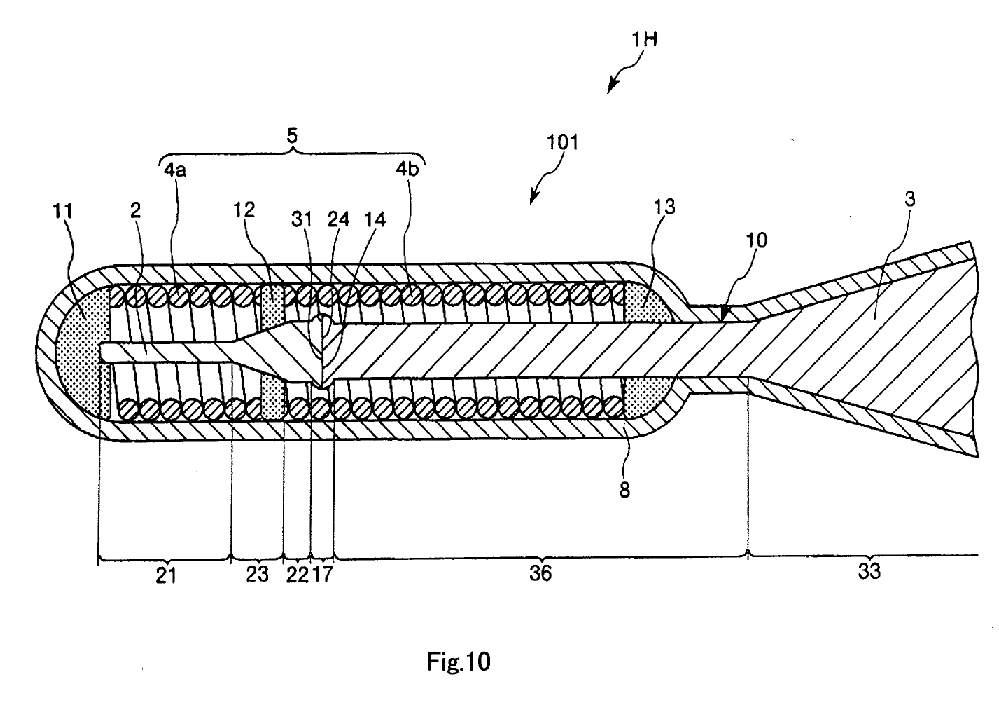

FIG. 10 is an enlarged longitudinal cross-sectional view of the distal portion of a wire body in a guide wire according to a ninth embodiment.

The following description of the ninth embodiment primarily discusses differences between this embodiment and the above-described embodiments. Features associated with the ninth embodiment that correspond to those in the previously described embodiments are designated with the same reference numerals used in the earlier embodiments, and a detailed description of such features is not repeated here.

This ninth embodiment is the same as the third embodiment above, except for the shape on the front and rear sides (the distal side and the proximal side) of the joint surface of the wire body.

In the ninth embodiment of the guide wire 1H shown in FIG. 10, the wire body 10 has a constant outer diameter on each of the front and rear sides of the joint surface 14 (projected portion 17). Specifically, in the guide wire 1H, a constant-outer-diameter portion 22 of the first wire 2 is formed on the distal side of the joint surface 14, and a constant-outer-diameter portion 32 of the second wire 3 is formed on the proximal side of the joint surface 14. In addition, the outer diameter of the constant-outer-diameter portion 22 of the first wire 2 is larger than the outer diameter of the constant-outer-diameter portion 32 of the second wire 3.

With the constant-outer-diameter portion 22 and the constant-outer-diameter portion 32 positioned on opposite sides of the joint surface 14, the flexural rigidity of the constant-outer-diameter portion 22 and that of the constant-outer-diameter portion 32 can be made equal (inclusive of substantially equal). This helps ensure that, when a distal portion 101 of the guide wire 1H is inserted into a curved blood vessel, the guide wire 1H can be smoothly curved even in the vicinity of the joint surface 14, so that the steerability of the guide wire 1H is enhanced.

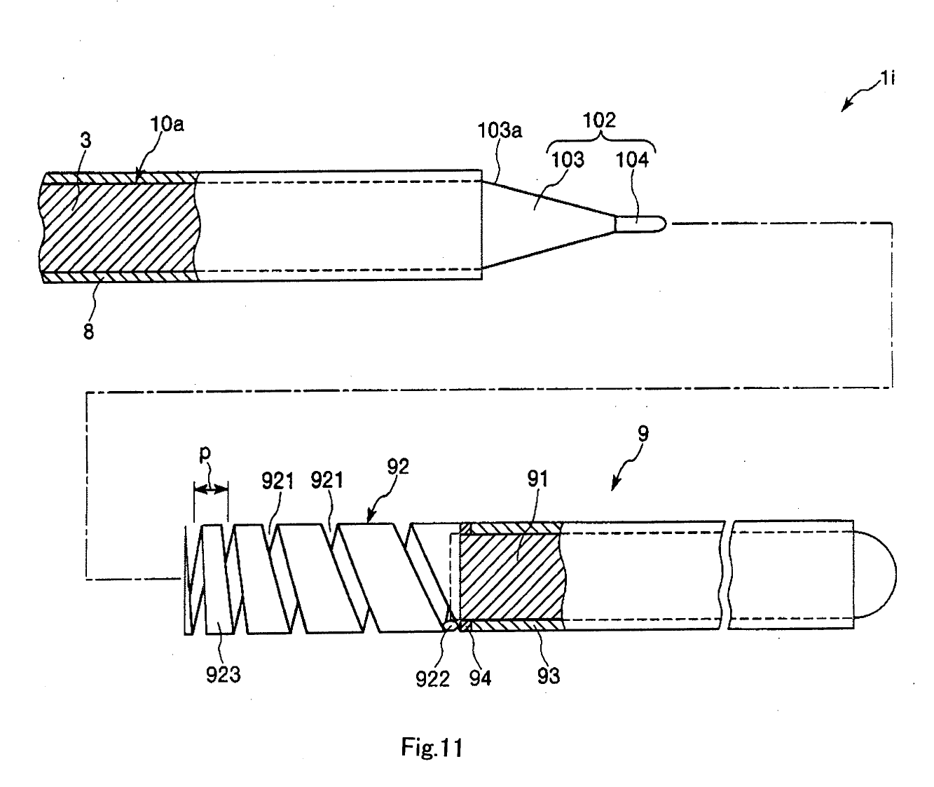

FIG. 11 is a partial longitudinal cross-sectional view of an extension wire of a guide wire according to a tenth embodiment.

The following description of the tenth embodiment primarily discusses differences between this embodiment and the above-described embodiments. Features associated with the tenth embodiment that correspond to those in the previously described embodiments are designated with the same reference numerals used in the earlier embodiments, and a detailed description of such features is not repeated here.

This embodiment is the same as the first embodiment above, except that the guide wire in this embodiment further includes the extension wire.

The guide wire 1i shown in FIG. 11 comprises the extension wire 9 which is detachably connected or attached to the proximal portion 102 of the wire body 10a.

Prior to describing the extension wire 9, the proximal portion 102 of the wire body 10a to which the extension wire 9 is to be connected is discussed.

The proximal portion 102 of the wire body 10a includes a tapered portion 103, and a projected or projecting portion 104 projecting from the proximal end of the tapered portion 103.

The tapered portion 103 is a portion in which the outer diameter is gradually reduced (in a tapered form) along the proximal direction.

The projected portion 104 is a portion projecting along the proximal direction from the end of the tapered portion 103. The projecting portion 104 has a constant outer diameter along the longitudinal direction of the wire, and the outer diameter is substantially equal to the outer diameter at the proximal end (minimum outer diameter) of the tapered portion 103. In addition, the proximal end surface of the projected portion 104 possesses a rounded shape.

The extension wire 9 is connected to the proximal portion 102 of the wire body 10a configured as described above. The extension wire 9 is connected to the wire body 10a (this condition will hereinafter be referred to as “the connected condition”), whereby the overall length of the guide wire 1i is enlarged. The overall length of the guide wire 1i in the connected condition is not particularly limited, but is preferably about 3,500 to 4,000 mm.

The extension wire 9 comprises a wire body portion 91 and a connecting portion (connecting pipe) 92 located at a distal portion of the wire body portion 91.

The wire body portion 91 includes a flexible or elastic filamentous member. The material constituting the wire body portion 91 is not particularly limited. By way of example, the same materials as those which can be used to constitute the second wire 3 can be used.

In addition, the wire body portion 91 is provided on its outside surface (outer peripheral surface) with a resin coating layer 93 covering the entirety of the wire body portion 91, or a part thereof. The resin coating layer 93 can be any of the materials discussed above for the resin coating layer 8. The resin coating layer 93 may be also be provided on the outside surface (outer peripheral surface) of the connecting portion 92, in addition to the outside surface of the wire body portion 91.

As shown in FIG. 11, the connecting portion 92, which is adapted to be connected to the proximal portion 102 of the wire body 10a, is fixed to a distal portion of the wire body portion 91. The fixing method in this case is not particularly limited. For example, a method in which a solder (brazing filler) 94 is used is adopted in the construction shown in FIG. 11.

The connecting portion 92 includes a pipe-like body. The overall length of the connecting portion 92 is not particularly limited, but is preferably about 20 to 70 mm.

The connecting portion 92 is provided with a spiral slit 921. In the connecting portion 92, the pitch (interval) p between the adjacent portions of the slits 921 is increased along the proximal direction. Incidentally, the pitch is set so that the ratio of the pitch p at the distal portion of the connecting portion 92 to the pitch p at the proximal portion of the connecting portion 92 is preferably in the range of 1.4 to 6.6, more preferably 2.8 to 3.3. The pitch p is gradually increased along the proximal direction so as to have the ratio in the just-mentioned range.

The slit 921 is formed over the entire range from the distal end to the proximal end of the connecting portion 92. This helps provide that the spiral condition of the wall portion of the connecting portion 92, i.e., the overall shape of the connecting portion 92, is reliably maintained.

A solder (brazing filler) is disposed at a proximal portion (terminal point) of the slit 921. By this, the overall shape of the connecting portion 92 is maintained more reliably.

The material constituting the connecting portion 92 is not limited to any particular material. For example, the Ni—Ti alloys mentioned in the description of the first wire 2 above can be used. By this, the connecting portion 92 can be relatively easily expanded and contracted in the radial direction (expanded/contracted in diameter), and the overall shape of the connecting portion 92 is maintained more securely. The inner diameter of the connecting portion 92 in its natural state is smaller than the outer diameter of a base portion 103a of the tapered portion 103.

To connect the wire body 10a and the extension wire 9 of the guide wire 1i, the proximal portion 102 of the wire body 10a is manually pushed into the connecting portion 92 of the extension wire 9. To accomplish this, the inner peripheral surface of a distal portion 923 of the connecting portion 92 is pushed by the outer peripheral surface of the base portion 103a of the tapered portion 103, whereby the distal portion 923 is enlarged in diameter. This helps ensure that the proximal portion 102 (tapered portion 103) of the wire body 10a is fitted in the connecting portion 92 of the extension wire 9. As a result, the proximal portion 102 of the wire body 10a and the connecting portion 92 of the extension wire 9 are connected to each other, whereby the guide wire 1i is put into the connected condition.

With the tapered portion 103, it is possible to achieve connection between the wire body 10a and the extension wire 9 according to the magnitude (size) of the inner diameter of the connecting portion 92.

The inside peripheral surface of the connecting portion 92 may have been subjected to a roughening treatment. By such a treatment, a multiplicity of microscopic recesses and projections are formed in the inside peripheral surface of the connecting portion 92 so that the connected condition can be prevented from being canceled unwillingly. In this embodiment, the connected condition of the extension wire 9 and the wire body 10a occurs solely by virtue of the mechanical fitting of the proximal portion 102 of the wire body 10a in the connecting portion 92 of the extension wire 9. That is, an adhesive or other connection method is not necessary and is not employed in this embodiment.

To cancel the connected condition, the wire body 10a and the extension wire 9 are pulled in opposite senses or directions. As a result, the fitting of the connecting portion 92 of the extension wire 9 and the proximal portion 102 of the wire body 10a is canceled, so that the extension wire 9 is detached from the wire body 10a.

The method for canceling the connected condition is not limited to the just-mentioned method (a pulling method). As an alternative, a method can also be employed in which the connecting portion 92 is rotated in such a direction that the connecting portion 92 is expanded in diameter, i.e., the connecting portion 92 is slackened.

The guide wire 1i in this embodiment is not limited to being used as a modification of the first embodiment, and its effects are displayed even in a general mode such as a mode in which the distal portion of the wire body 10a is covered with a coil.

While the guide wire here has been described referring to the embodiments shown in the drawings, the invention is not limited to such embodiments. Components of the guide wire can be replaced with other arbitrary components which exhibit the same or equivalent functions. Also, arbitrary structures may be added.

A guide wire can also be constructed to include features from two or more of the above-described embodiments.

For example, a construction may be adopted in which the boundary portion between the first wire and the second wire in the fourth embodiment includes a joint surface, like the boundary portion between the first wire and the second wire in the first embodiment. In this case, a projected portion may be formed in the vicinity of the joint surface like the projected portion in the third embodiment.

The guide wires shown in FIGS. 1-10 may further include an extension wire such as the one possessed by the guide wire shown in FIG. 11.

The purpose for which the guide wire disclosed here is used is not limited to the use in the above-mentioned PTCA. The guide wire can be used, for example, in angiography, endoscopic procedure, etc.

Other examples of the flexible member include a resin layer including a resin material such as polyurethane.

While the magnitude relationship between the length L2 and the length L3 is L2<L3 in the first embodiment and L2>L3 in the second embodiment, these relationships are not limitative. For example, a relationship of L2=L3 may also be adopted.

The principles, embodiments and modes of operation have been described in the foregoing specification, but the invention which is intended to be protected is not to be construed as limited to the particular embodiments disclosed. The embodiments described herein are to be regarded as illustrative rather than restrictive. Variations and changes may be made by others, and equivalents employed, without departing from the spirit of the present invention. Accordingly, it is expressly intended that all such variations, changes and equivalents which fall within the spirit and scope of the present invention as defined in the claims, be embraced thereby.

What is claimed is:

1. A guide wire comprising:
a wire body possessing an outer periphery and comprised of a first wire and a second wire;
the first wire being positioned on a distal side of the second wire, the first wire being made of a Ni—Ti alloy;
the second wire being positioned on a proximal side of the first wire, the second wire being made of a material higher in rigidity than the material constituting the first wire;
the first wire and the second wire being connected to each other;
a flexible member covering the outer periphery of at least a distal side portion of the wire body, the flexible member exhibiting flexibility and being comprised of a tubular body having an inner diameter;
a boundary portion of the wire body between a proximal portion of the first wire and a distal portion of the second wire is located inside the tubular body of the flexible member;
the boundary portion of the wire body comprises a projected portion projecting in a radially outward direction of the wire body, the projected portion possessing an outer surface; and
an outer diameter of the projected portion is smaller than the inner diameter of the flexible member so that the outer surface of the projected portion is spaced from an inner surface of the flexible member.

2. The guide wire as set forth in claim 1, wherein the flexible member surrounds a portion of the first wire and a portion of the second wire so that the portion of the first wire and the portion of the second wire are covered by the flexible member, the portion of the first wire covered by the flexible member being longer than the portion of the second wire covered by the flexible member.

3. The guide wire as set forth in claim 1, wherein the flexible member surrounds a portion of the first wire and a portion of the second wire so that the portion of the first wire and the portion of the second wire are covered by the flexible member, the portion of the first wire covered by the flexible member being shorter than the portion of the second wire covered by the flexible member.

4. The guide wire as set forth in claim 1, wherein the flexible member is comprised of a coil formed as a spirally wound filamentous member.

5. The guide wire as set forth in claim 1, wherein a wall portion of the tubular body is provided with a groove and/or a slit.

6. The guide wire as set forth in claim 1, wherein the flexible member is comprised of two separate component parts arranged along a longitudinal extent of the wire body.

7. The guide wire as set forth in claim 6, wherein each of the two component parts is a coil formed as a spirally wound filamentous member.

8. The guide wire as set forth in claim 6, wherein one of the two component parts is a coil formed as a spirally winding filamentous member, and the other component part is a metal tubular body.

9. The guide wire as set forth in claim 6, wherein the two component parts are each comprised of a metallic material, the metallic material comprising one of the two component parts being the same metallic material, or different metallic material, relative to the metallic material comprising the other of the two component parts.

10. The guide wire as set forth in claim 6, wherein one of the two component parts is a proximal component part located on a proximal side of the other component part, and the boundary portion is covered by the proximal component part.

11. The guide wire as set forth in claim 1, wherein the first wire has a proximal end face and the second wire has a distal end face, the proximal end face of the first wire and the distal end face of the second wire being are joined to each other in abutting relation.

12. The guide wire as set forth in claim 11, wherein the proximal end face of the first wire and the distal end face of the second wire are welded to each other.

13. The guide wire as set forth in claim 1, wherein a proximal end portion of the wire body is tapered so that its outer diameter is smaller in a proximal direction, and further comprising an extension wire connected to and manually separable from the tapered proximal end portion of the wire body, the extension wire comprising a connecting portion engaging an outer peripheral surface of the tapered proximal end portion of the wire body and a wire body portion extending proximally of the connection portion, the connection portion comprising a spiral slit.

14. A guide wire comprising:
a wire body comprised of a filamentous first wire and a filamentous second wire, the wire body possessing an outer periphery;
the second wire being disposed proximally of the first wire;
the first wire being comprised of an alloy including Ni and Ti;
the second wire being comprised of a material higher in rigidity than the material constituting the first wire;
the first and second wires being connected to each other;
a flexible member covering the outer periphery of a portion of the wire body on at least a distal side of the wire body, the flexible member possessing flexibility;
a proximal portion of the first wire and a distal portion of the second wire forming a boundary region of the wire body that is located inside the flexible member;
a connecting member providing a connection between the proximal portion of the first wire and the distal portion of the second wire; and
at least a portion of the connecting member is supported relative to the flexible member.

15. The guide wire as set forth in claim 14, wherein a distal portion and/or a proximal portion of the connecting member is fixed to the flexible member by fixing material so that the connecting member is supported relative to the flexible member.

16. The guide wire as set forth in claim 14, wherein an intermediate portion of the connecting member is fixed to the flexible member by fixing material so that the connecting member is supported relative to the flexible member.

17. The guide wire as set forth in claim 14, wherein the distal portion of the second wire and the proximal portion of the first wire are constant outer diameter portions possessing a constant outer diameter; the constant outer diameter proximal portion of the first wire or the constant outer diameter distal portion of the second wire adjoining a stepped section possessing a varying outer diameter, and further comprising fixing material fixing the flexible member to the stepped portion and to either the distal end or the proximal end of the connecting member.

18. The guide wire as set forth in claim 14, wherein the distal portion of the second wire and the proximal portion of the first wire are constant outer diameter portions possessing a constant outer diameter, the constant outer diameter proximal portion of the first wire adjoining a first stepped section possessing a varying outer diameter, the constant outer diameter distal portion of the second wire adjoining a second stepped section possessing a varying outer diameter, and further comprising a fixing material fixing the flexible member to the distal end of the connecting member and the first stepped portion, and a second fixing member fixing the flexible member to the proximal end of the connecting member and the second stepped portion.

19. The guide wire as set forth in claim 14, wherein a proximal end portion of the wire body is tapered so that its outer diameter is smaller in a proximal direction, and further comprising an extension wire connected to and manually separable from the tapered proximal end portion of the wire body, the extension wire comprising a connecting portion engaging an outer peripheral surface of the tapered proximal end portion of the wire body and a wire body portion extending proximally of the connection portion, the connection portion comprising a spiral slit.

20. A guide wire comprising:
a wire body possessing an outer peripheral surface and comprised of a filamentous first wire and a filamentous second wire, both the first and second wires possessing an outer peripheral surface;
the second wire being positioned proximally of the first wire;
the first wire comprising a proximal end portion terminating proximally in a proximal end face;
the second wire comprising a distal end portion terminating distally in a distal end face;
the proximal end portion of the first wire possessing an outer diameter that is constant to the proximal end face to define a constant outer diameter proximal end portion of the first wire;
the distal end portion of the second wire possessing an outer diameter that is constant to the distal end face to define a constant outer diameter distal end portion of the second wire;
the first wire being comprised of an alloy including Ni and Ti;
the second wire being comprised of a material different from the material constituting the first wire;
the material constituting the second wire being different in rigidity than the material constituting the first wire;
the proximal end face of the first wire abutting and being joined to the distal end face of the second wire;
a flexible member possessing a distal end, a proximal end, and an intermediate portion between the distal and proximal ends;
the flexible member covering the outer peripheral surface of a portion of the wire body so that an entirety of the constant outer diameter proximal end portion of the first wire is positioned inside the flexible member and at least a part of the constant outer diameter distal end portion of the second wire is positioned inside the flexible member;
the outer peripheral surface of the constant outer diameter proximal end portion of the first wire being spaced from an inner surface of the flexible member;
the outer peripheral surface of said part of the constant outer diameter distal end portion of the second wire being spaced from the inner surface of the flexible member;
a first fixing member fixing the distal end of the flexible member to the wire body;
a second fixing member fixing the proximal end of the flexible member to the wire body;
a third fixing member fixing the intermediate portion of the flexible member to the wire body; and
a length of the constant outer diameter proximal end portion of the first wire that is positioned inside the flexible member being different from the length of said part of the constant outer diameter distal end portion of the second wire that is positioned inside the flexible member.

21. The guide wire as set forth in claim 20, wherein the first wire comprises a stepped section in which an outer diameter of the first wire varies, the stepped section being located distally of the constant outer diameter proximal end portion of the first wire, the first wire further comprising a constant outer diameter distal portion possessing a constant outer diameter and located distally of the stepped section, the stepped section and at least a part of the constant outer diameter distal portion being covered by the flexible member.

22. The guide wire as set forth in claim 20, wherein the second fixing member fixes the proximal end of the flexible member to the constant outer diameter distal end portion of the second wire.

23. The guide wire as set forth in claim 20, wherein the flexible member comprises only a spirally wound coil or a tubular member provided with at least one of slits or grooves.

24. The guide wire as set forth in claim 20, wherein the flexible member comprises a distal side flexible member and a proximal side flexible member, the proximal side flexible member being positioned proximally of the distal side flexible member, the distal side flexible member having a proximal end that is fixed to a distal end of the proximal side flexible member by the third fixing member.

25. The guide wire as set forth in claim 24, wherein the proximal end face of the first wire is welded to the distal end face of the second wire at a weld region, the weld region being covered by the proximal side flexible member.
